(19) United States
(12) Patent Application Publication
(10) Pub. No.: US 2006/0173708 A1
(43) Pub. Date: Aug. 3, 2006

(54) System and method for providing health care

(75) Inventors: David Vining, Andover, MA (US); Bert R. Vining, Andover, MA (US); Craig A. Walker, Austin, TX (US); Neil O. King, Cave Creek, AZ (US)

Correspondence Address: NUTTER MCCLENNEN & FISH LLP, WORLD TRADE CENTER WEST, 155 SEAPORT BOULEVARD, BOSTON, MA, 02210-2604, US

(73) Assignee: Circle of Care, Inc., Andover, MA (US)

(21) Appl. No.: 11/046,091

(22) Filed: Jan. 28, 2005

(51) Int. Cl. G06Q 10/00; A61B 5/00

(52) U.S. Cl. 705/2; 600/300

(57) ABSTRACT

A method of providing health care by establishing a predetermined patient population grouped by geographic regions where the patient population has access to a traveling healthcare professional that may travel to the patient's location. The healthcare professional evaluates and examines the patient using a plurality of technologies including a microprocessor and memory storage that is coupled to, or interfaced with, audio/video/data transfer and communications systems, medical devices, and other vital measurement devices that the physician located at the physicians' center uses to assess and consult the healthcare professional who is onsite with the remote patient. During the exam the healthcare professional uses audio/video/data communications for interconnecting the healthcare professional with a physician at the physicians' center. The physician at the physicians' center conducts a virtual house call to help maintain the physician-patient relationship, evaluate the collected information, and instruct the healthcare professional during the physician's examination and evaluation. The physician having access to the patient's health records, family medical history, present medications, prior allergic reactions and any adverse drug interactions or contraindications, assesses, and renders a diagnoses and treatment plan back to the healthcare professional. The healthcare professional under the delegation of the physician treats the patient under the physician's instructions. The physician may prescribe medication and transmit the prescription to a pharmacy. The physician updates the patient's health record with the collected information, diagnosis, physician orders, treatment plan, patient input information, and clinical notes. The method also provides for a patient to obtain medical information without the need for a healthcare professional's visit. The method also provides many illustrations that allow for the assessment, treatment, diagnosis, and sharing of medical information and care to be provided to the patient at the patient's location, and to authorized healthcare professionals.

### FIELD

The invention relates to a system and method of providing health care to a patient or patient population without the need for the patient(s) traveling to a specific location such as a hospital or physician's office to receive medical care. Specifically, the patient as a member of a predetermined patient population, or health plan, who resides in a geographic region seeks personal healthcare services in their domain or location by contacting the health plan's call center. The call center, having access to the patient's health records reviews the patient's past medical history, evaluates their family medical history, communicates with the patient, learns of the patient's past and present medications, investigates if the patient has experienced any past allergic reactions or drug interactions, and triages the patient using predetermined algorithms, clinical protocols, and appropriate health aides. Based on the algorithms, protocols, and clinical determination, the triage personnel at the call center may provide general health advice over the telephone or dispatch a healthcare professional to the patient's location for a virtual house call. Once at the patient's location, the healthcare professional may employ a plurality of technologies which includes a display unit for viewing multimedia, a microprocessor having a memory storage device coupled to a audio/video communication system, data communication system, medical devices, and vital measurement instruments to assess the patient's health status and collect patient-specific vital readings that can be transmitted in a synchronous or asynchronous manner to a consulting physician at a physicians' center. The physician, upon review of the patient's medical history and health record, and upon reviewing the patient's family medical history, learning of patient's past or present medications, contraindications and investigating if patient has experienced any past allergic reactions or drug interactions, establishes a legitimate patient-physician relationship and is capable of performing a remote medical consultation, or virtual house call, of the patient using the information that the healthcare professional collects while with the patient. The physician located at the physicians' center reviews existing health records and prescription history and delegates the remote healthcare professional on the appropriate treatment throughout the virtual house call. The physician having an established patient-physician relationship may prescribe medications, durable medical equipment, issues medical orders, records the patient encounter into the patient's health record, and may share this information with the patient's primary care physician, specialist, or other appropriate medical professional. Another embodiment of this invention involves the patient contacting the call center simply to receive general health and medical information over the telephone. In yet another technological rendition, the patient upon contacting the call center can be directed to a physician who, upon review of the patient's health record and having established a physician-patient relationship, provides consultation to the patient over the telephone. Alternatively, a patient can call the call center using a telephone and the triage personnel at the call center may determine that emergent care is required and the call center may dispatch emergency medical services to the patient's location, or the call center may direct the patient to travel to an appropriate health facility for a higher-level of care. In each of these implementations the encounter is recorded into the patient record system and made available when required to additional health providers.

### BACKGROUND

Health care has been an integral part of American society since early colonization. As people communed together either for protection, survival, or commerce, health care has been a component to everyday life. It has only been in the last several decades, however, that the integration of healthcare and wellness services has been promoted to the forefront of our society's public concern. This has mainly resulted in part from the continued aggregation of people and their demand to utilize available healthcare services from local providers. The increased demand for healthcare and wellness services has brought about some complexities that have forced many managers of hospitals, health service organizations and health systems to further explore methods of contracting and financing the delivery of these services. Health service organizations may be classified as entities that offer minimum organizational structure to provide the delivery of health services to the consumer, whether the purpose of the services is preventative, acute, restorative, or palliative. Coupled with the consumer's demand for health care, state and federal regulatory provisions either require or induce health service organizations to provide these services in an efficient, seamless manner. This emphasis to deliver efficient health services and control the associated costs incurred has encouraged many healthcare organizations to integrate the delivery of primary care and specialty services across a network of physicians and health service organizations in order to provide a continuum of healthcare and wellness services to a regional population.

Integrating services is extremely challenging and costly in lieu of dynamic changes resulting from governmental and environmental influences. These challenges, while dynamic, have forced many healthcare organizations to further explore ways to provide and deliver healthcare and wellness services for optimum performance at the most affordable cost. In the early days of modern medicine, physicians provided patients medical and surgical services at the patient's location. This approach to medicine provided a “personal” approach to receiving the medical care. In traditional medicine, patients received “house calls” by the physician. Generally, when a patient contacted a physician, the physician traveled to the location of the patient, and the physician would examine and treat the patient at the patient's location. If the physician deemed it necessary the patient would be moved to another location for treatment. The patient seldom left the comfort of their home, or location, to receive their health care from a physician. As the quality of care and the demand for a standardized systematic approach for delivering medical services heightened, the role of the hospital became pervasively important and the “personal” approach of health care became less and less pervasive.

The desire to congregate extremely sick people for isolation from the healthy population, and the impact from formal medical training greatly affected the role of the hospital and the location where medical care was administered or received. By the mid-19^{th} century, technological advancements in public health and medical knowledge permitted physicians and health care workers to perform surgical procedures in a more sanitary environment. These advancements in medical practices and sanitation encouraged a centralized workplace in order to use this technology to perform these procedures; thus helping heighten the importance of the hospital as a centralized place to providing these medical services. This evolution allowed people to obtain medical care from a specific facility, rather than from a single physician providing the care at the patient's home, or location. As a result, the method and style of delivering medical care to the patient dramatically changed.

With the advent of managed care, the desire for health service organizations to contain costs also greatly impacted the “personal” approach to the delivery of medicine. Equally, the emergence of the consumer driven health plans in the market is further fueling consumer choice in the type of healthcare coverage desired by enabling the consumer to play a more critical role in determining the types and level of desired healthcare services they are willing to pay for. The existence of the consumer driven health plan is directly related to consumer frustration that is resulting from poor experiences with the level of care consumers receive for the cost for their care.

Today's modern healthcare system is being affected by the confluence of American consumerism and the increased costs to providing care. These influences are straining traditional medical settings in how they provide basic clinical services while at the same time maintaining their existing level of services. As a result, many health plans and government-sponsored programs are beginning to place an increased burden on the consumer to help finance overall health services. Federal initiatives like the Modern Prescription Drug Benefit Plan and the introduction of consumer-driven health benefit plans, as well as changes to state-sponsored programs for covering children health insurance, foreshadows the consumer picking up a larger percentage for the total cost to their care. In addition, the historical paternalistic model to never question the directive of the physician is waning as consumers become further empowered with available information. Additionally, consumer discontent is growing as a result of the continued prevalence of medical malpractice, rising healthcare costs, and substandard clinical care. As a result, consumers are beginning to publicly voice more of their concern to have more involvement and control in determining their overall covered health services in lieu of their investment. This heightened consumer awareness is further compounded by consumers being more informed and prone to educate themselves on understanding their health status and medical condition. For today, the consumer has access to online medical research and healthcare portals, like WebMD and other Internet websites that provide information related to a myriad of health conditions. Consumers are demonstrating they are willing to pay more out-of-pocket money for their health services if they see value in the level of services they are receiving. Wait times in physician office clinics, hospitals, and emergency rooms are increasing because these healthcare providers are required to provide the same level of service for less money. These factors are constraining modern medicine and creating consumer experiences that are greatly impersonal. As a result of these compounding factors, a novel approach is designed to bring back the traditional physician “house call” and develop a more personal approach to providing and receiving patient care through the development of alternative delivery system and the use of modern technology.

Today, if an individual seeks medical care that person will contact their physician, call an ambulance, or go to the emergency room. If a person contacts their physician to schedule an appointment, the patient may wait days, or weeks, before seeing the physician. If a patient desires immediate medical attention, they may require transportation to a medical facility, or hospital emergency room, all in the hope to have a physician examine and treat them in a timely fashion. It is not uncommon that when someone seeks non-emergent care from a hospital's emergency room, or general medical facility, they may wait several hours or more before they receive medical attention and can result in creating frustration to the patient and their family members. The patient may also find it difficult to find a facility with an available physician to even render the medical care because it is late at night, or very busy. Not all hospital emergency rooms and clinics are open to the public around the clock; they may be open during the day and closed during the night. Hospital emergency rooms and clinics have limited space and resources and it is not uncommon for a person seeking medical attention to have a negative experience from seeking care.

As a result of these adverse experiences, patients may not be satisfied with the level of care they receive. To compound this dilemma, patients may seek care from healthcare providers that do not possess any prior history of the patient's medical condition or medication history, and the physician is required to assess, diagnose, and treat the patient without the aid of this information. This type of patient encounter often increases the time to assess and treat the patient, and it may consume several hours of waiting time and further exasperate the patient's experience.

The entire experience from first traveling to the medical facility, getting treated and diagnosed by a physician can be a time consuming, unconformable, and frustrating proposition to the patient. In addition, the physician's time to locate and read thorough the patient's medical record can also add to the wait and frustration to both the patient and physician. Compounding the amount of time it takes for the patient to receive care, the physician may issue orders, prescribe medications or durable medical equipment, that requires the patient to possibly travel and seek out this type of treatment. For example, the patient may require transportation to a pharmacist to have their prescription filled and be forced to wait until the prescription is filled. In other cases, the patient contacts a pharmacist and receives the prescription or durable medical equipment by postal mail or delivery service. Equally, the patient may require diagnostic testing for further analysis, like medical imaging and blood analysis, and this may require technologies located at other facilities for the patient to obtain. As a result, the contemporary healthcare experience is de-personalized. In order to rectify the personal approach to the healthcare experience, the inventor recognizes that the “personal” approach to delivering modern medicine has nearly absolved from common practice, and has a new paradigm for offering the traditional “house-call” by employing technological innovation through the novel invention of the virtual house call. With a virtual house call, a patient can receive personal health care in their home, or at their location, twenty-four hours a day from physicians and medical personnel. The inventor speculates that this personal approach to delivering health care will help increase patient satisfaction and provide timely care in a personal manner.

It is well know in the arts that many inventors have attempted to solve these problems of providing timely healthcare. One such way is a method of remote patient management and possible diagnosis using a remote monitoring device that collects and transmits vital readings of the patient's health status to a central server. Typically, this is accomplished by leaving monitoring equipment at the patient's home which is capable of receiving and transmitting patient vital readings and data that represents patient information, such as a physiological characteristic or vital bodily function of the patient. This patient information is transmitted to a central server where a clinician or physician reviews the data. This process is accomplished by communicating the remote patient's physiological condition and health status readings to the selected clinician or physician via an electronic communications device. While this method achieves the remote collection of patient data, the inventor views this approach as costly and impersonal.

It is also well know in the arts that systems and methods for providing on-line healthcare are common. These systems utilize the Internet or on-line network communications modality for data transfer to receive patient symptom and health status information. The process involves a person, like a patient, requesting health care from a healthcare provider or healthcare provider group. The healthcare provider or healthcare provider group generates a health assessment based on predetermined algorithms and related symptom informational databases and provides an on-line opinion over the Internet, or the on-line network communications modality, with the person requesting the healthcare. These systems may also include a system and method for ordering prescriptions over an on-line network communications modality, transmitting the health assessment information to a patient's personal physician over the on-line network communications modality, and providing a physician's referral for the patient. Again, the inventor finds this system inadequate and impersonal for individuals who prefer “personal” medical care from a physician. In addition, these claims describing approaches to on-line healthcare do not permit for establishing the physician-patient relationship. Generally, these methods only provide the patient with limited information and the patient therefore must travel to seek care from a clinician, or physician at a healthcare facility, such as a physician office or hospital.

Yet another prior art solves this problem by providing to a medical organization, via a communications network, a variety of medical data measured from a patient staying at home. These systems include using a plurality of remote patient monitoring units set-up within the homes of patients and these remote patient monitoring units are connected using a network communications system that may include a Public Telephone Switch Network (PSTN), Digital Subscriber Lines (DSL), wireless communications, Bluetooth, and cellular communications that transmit data to a centralized medical organization that is comprised of a internal local area network (LAN) interconnecting these units via communication lines to appropriate clinicians or physicians. The medical data is transmitted from the home units to centralized medical organization's units via hardware or software interface units that correspond to various measurement apparatuses and communication devices. A communication device such as a modem which allows for bi-directional communications between each of the home units and the medical center allows information to be transferred and exchanged on a synchronous, real-time basis, or asynchronous, store & forward basis. These remote patient monitoring units can obtain a plurality of medical and health related data elements such as blood pressure, pulse wave, electrocardiogram, cardiac auscultations, pulmonary auscultations, body temperature, body and chest cavity auscultations, vital readings, and other biometric and physiological readings and transmit such a plurality of medical and health data via network communications systems to a central unit at the medical center where physicians or clinicians may receive and monitor the patient-specific data. Again, the inventor finds this approach impersonal and places much of the locus for equipment set-up and monitoring as a responsibility of the patient.

The contemporary way of providing health care at a health facility, or physician clinic, has been found by the inventor to be time consuming, costly, impersonal, and frustrating to the patient. Patients who seek non-emergent care in a hospital emergency room often queue for hours and hours before being attended by a clinician. Accordingly, scientific research validates that a patient's medical experience has a large bearing on their recovery and future health status. The inventor finds that the prior art of a remote set-up home patient monitoring unit for and the use of on-line healthcare removes the personal approach and professional hands-on intervention that many patients desire. However, the present invention and its embodiments does enable a hands-on health professional, who travels to the patient residing within the geographic regional health service area, to deliver health care in a personal manner to the patient at the patient's location while at the same time helps maintain the physician-patient relationship which is made manifest through the use of audio/video/data communications between the health professional and the physician conjoined with the physician's review of the patient's health record. The present invention and its embodiments enables the health professional to use a device known by the inventor as the 21^{st} Century Doctor's Bag, a device that is comprised of a plurality of technologies including a display unit, a microprocessor having a memory storage device coupled to medical devices, telecommunications systems, audio and video communication systems, and data communication systems which enables healthcare providers to provide appropriate medical services including emergency medical services. The implementing devices of the mobile, portable unit, known by the inventor as the 21^{st} Century Doctor's Bag includes devices such as portable digital imaging units, digital and analog monitoring devices, ultrasound and digital radiography systems, as well as microscopic and spectroscopy-related imaging devices that provide healthcare professionals, who are onsite with the patient, the ability to provide remote diagnostic assessments and treatments while in constant communication with the physician and all the while having an individual, like the Healthcare Advocate, who maintains constant, personal monitoring of the entire patient's encounter. The 21^{st} Century Doctos Bag, which is a mobile, portable unit, transmits data and relevant information that provides a physician patient information and vitals data associated with the remote, virtual house call. This novel method of assessing and treating the patient through the virtual house call helps ensure the physician-patient relationship, and it helps reduce adverse situations arising from queuing, which can further compound and augment the patient's illness or condition, by enhancing the patient's personal experience by providing the health service at the patient's location.

Today, patient satisfaction outcomes reveal compounding frustration from the experience of being responsible for setting-up remote patient monitoring equipment or seeking on-line health consults since these scenarios are entirely too impersonal and often intimidating for some individuals. With today's emerging technology and the general frustration of individual patients who spend a lot of time waiting and traveling to a medical facility, becoming frustrated with the lack of personal care they are provided with, the time has come for a better, more personal approach to providing health care at the patient's location and in a manner that ensures the establishing of the physician-patient relationship by creating a new paradigm for the traditional physician's house call—the 21^{st} Century Virtual house call through “The Connected House Call.”

### SUMMARY

The present invention provides a system and method of providing an alternative healthcare delivery system to the traditional, historical setting, such as a physician's office, clinic, hospital, tertiary hospital, and local health care facility. The invention accomplishes the provision of providing an alternative healthcare delivery system by employing technology in novel methods that ensures the establishment of a legal, traditional face-to-face physician-patient relationship. In addition, the present invention accomplishes the provision of providing an alternative healthcare delivery system through the establishment of a predetermined patient population entailing detailed member, or subscriber, enrollment, and the provision of health care and member services embodying the creation of an exclusive member-service health plan.

Members who subscribe to this alternative healthcare delivery system and its services constitute the embodiment of a health plan, which is implemented through a comprehensive member enrollment process whereby information is collected that is necessary in providing general medicine and acute care and in helping establish a legal patient-physician relationship. The process involves a system and method by which a patient enrolls in a health plan and healthcare providers deliver care. Patients enroll to participate within geographic areas and receive member services from the health plan, which is similar to a health service organization, managed care plan, or health benefit plan, or an entity organized to provide a patient population with health benefits. During patient enrollment, the health plan obtains information critical to the establishment of a physician-patient relationship including but not limited to the patient's past medical history, current medical history, family medical history, present medications, and any prior allergic reactions and contraindications; this collected information will aid in the establishment of a physician-patient relationship along with information that constitutes the patient information that is used by the health plan in managing the patient's member benefits and healthcare services.

The patient information is recorded into an electronic medical record, or patient record, whereby the physician uses this information in establishing the physician-patient relationship, and embodiments of this process constitute the patient's intake information for enrollment into the health plan and therefore establish them within a predetermined patient population. Patients within a predetermined patient population may be categorized based on member services and health status. The patient pays the health plan for member services, particularly personal health services that may be accessed twenty-four hours a day, seven days a week. The patient, as a member of a predetermined patient population, subscribes to participates in receiving health and medical care by first enrolling in the member services of the health plan. The present invention's implementation of a health plan utilizes a medical record system that includes individual information on each patient that is used in the plan of care of the individual patient. A subscribing patient either accesses health care as part of at least a patient population group, which includes a group of people residing in similar geographic areas, or the patient accesses health care from an international or domestic location. Embodiments of this patient's access to health care occur through the direct patient communication with a physician, healthcare provider, or other individual using an audio and video communication system, or communications system. The patient may communicate with the present invention's embodiment of a health plan through the use of a communication system, such as audio and video communications, or telephonic or cellular communication systems, such that the patient converses with a healthcare advocate, such as a person designated as the subscriber's personal point of contact with the health plan. In addition, the patient may be routed to an alternative medical service provider through a facilitated hand off whereby the present invention directs and facilitates the patient's communication and provision of care to an outside healthcare provider.

In the event that the patient is outside the health plan's normal geographic health service area, the patient may simply communicate via a telephonic visit, such as a phone call with a designated individual with the health plan, who will instruct and communicate the patient concerning their member services and route them to an appropriate healthcare provider for their provision of health care. Patient populations may be predetermined based on health status, household, or other classification such as by diagnosis code or disease condition. An embodiment of this process includes the use of an electronic medical record that permits healthcare providers to record, store, archive, access, and update patient information and episodic, clinical information into a software system. Patient information is inputted into this type of embodied system using a standard manual entry, like a keyboard, or electronic data interchange. The electronic data interchange includes automated processes of obtaining personal and general information using technology such as voice recognition, bar-coding, radio frequency identification, personal identification, servers, computers, and personal digital assistants. Further embodiments of the present invention's type of data that results from electronic data interchange processes may include data that can be used and analyzed by additional business applications and processes that include data mining and statistical analysis that create additional vertical markets, such as offering additional member services including life insurance, claims adjudication, pharmaceutical marketing, clinical imaging databases, global access to electronic health record data, disease state management, chronic care management, and other direct and indirect business processes and clinical applications.

Embodiments of the present invention's implementation of a health plan enables a patient population, located within a geographic region, or health service areas, to not have to travel to a medical facility to receive their personal care. More particularly, an embodiment of this invention relates to a manner in which patients who are subscribers, such as the patient population group, to receive health services more effectively, efficiently, and in a more personal manner while maintaining a physician-patient relationship.

Using the embodiments and implementations of this invention will allow the user to construct and service patients who desire a more personal approach to receiving contemporary medical care. Embodiments and implementations of this present invention will allow the user to experience the services of a health plan having the same, if not better, patient satisfaction outcomes and clinical care as with previous contemporary methods. Moreover, embodiments and implementations of the present invention will allow the user to construct and use a patient healthcare system that is more personal, efficient, and effective without sacrificing patient care, nor compromising the clinician's or physician's ability to treat and diagnose the patient's condition.

The health plan's available services reflect the market trend to develop consumer-driven health plans in response to the changes in market pressure by the consumer to direct and package health services towards consumer demand for more personal care. This invention is directed toward creating and maintaining a system and method for providing health care to a patient, or a patient population. The invention directs care to the patient, or patient population, who may not be necessarily located in a traditional medical environment, using telecommunications and technology to provide the traditional essence of the face-to-face, physical exam. The invention enables a healthcare provider located at a physicians' center to simulate the traditional face-to-face patient encounter by directing the remote patient's plan of care using a technological communications systems, like audio/video conferencing systems and devices that permit the transmission of digital data and analog data. The embodiment of this form of communication and modality in which patient care is directed constitutes the virtual house call.

Upon establishing a call center, the health plan receives calls from patients within the patient population or from patients outside a health service area. The call center receives that patient's call and records the patient's information during the call center's intake process. The patient directs and communicates their need for medical care to the health plan's call center, and the patient's plan of care is triaged based on the patient's communicated intake information. The call center's triage process involves routing callings based the severity of the patient's illness and the intensity of the patient's service, as well as through the use of the health plan's patient management protocols. The patient communicates with clinical personnel or a healthcare advocate that aids in the collection and dissemination of medical information and may also provide the patient with information on events as they occur, mostly remaining in communication with the patient as events occur or as long as the patient wishes. The clinical personnel may include a healthcare advocate, or individual who is assigned to the patient to be their personal point of contact in accessing and receiving subscribed member services, and they can also help the patient through a medical event by aiding in comforting the patient. The healthcare advocate communicates with the patient on general subscriber information and patient-specific healthcare matters. In addition, the healthcare advocate records patient input information using a manual or automated processes, coordinates the patient encounter information with the appropriate healthcare professional, such as a physician, provides aid to the patient if necessary, monitors the patient's medical condition, and follows the patient's health services, ongoing treatment and health status.

The call center personnel may arrange and dispatch a healthcare professional to travel to the patient's location and may notify the patient's healthcare advocate and physician of the impending patient visit. Upon completing the telephone triage and using appropriate algorithms, the call center updates and records the patient's health record and business records to reflect the recent patient-encounter.

The healthcare advocate may follow-up with the patient at a later date subsequent to the patient receiving care to assure the patient's satisfaction and any of the patient's subsequent medical needs. In one illustration of this invention, a healthcare professional travels to the patient's location, examines, and collects a number of patient-specific vital readings and parameters including but not limited to any input from the patient such as patient described symptoms and observations. The healthcare professional can obtain and transmit any collected, patient-specific information to a physician at the physicians' center. In addition, the healthcare professional along with the patient can conduct an audio/video conference with the physician located at the physicians' center who can reciprocate with communications back to the remote healthcare professional. Information taken or received from the patient, or information obtained about the patient, can be patient-encounter data. In an other implementation of this invention the patient care supplied by the healthcare professional can take any of a number of forms, including but not limited to physically examining the patient using visual or hearing scenes, for example listening to auscultations of the abdomen and chest cavity (example, the patient's heart beat), or visually examining the patient's skin condition. Each these functions of the healthcare professional can take place under the direction of a physician using a communications system where embodiments include audio/video conferencing systems and the electronic transmission of data.

The healthcare professional at the patient's location may use a variety of techniques to examine the patient. First, the healthcare professional would obtain and collect patient information such as asking the patient questions related to their physical, behavioral, and mental status. Next the healthcare professional would also make his or her own sensatory evaluation such as visual, touch, or listening to bodily auscultations. The healthcare professional is in communication with a physician who can direct and instruct the healthcare professional. The healthcare professional uses a device known by the inventor as the 21^{st} Century Doctors Bag, or similar facsimile, which is comprised of portable technology that connects with a plurality of examination equipment, diagnostic equipment, communication systems, and medical devices that may be adapted to relay information back to the physician located at the physicians' center. The portable technology is a device that is comprised of at least a display unit for viewing multi-media, such as a color monitor, black and white monitor, or other viewing apparatus, and the device contains a microprocessor having a memory storage device, similar to random access memory or flash memory storage. Examples of the plurality of examination equipment and medical devices that the healthcare professional may use include, but are not be limited to, a thermometer to check the patient's body temperature, a digital camera capable of communicating images back to the physician, an ear, nose and throat device coupled with attachments to view the patient's ear, eye, throat, or skin, a stethoscope to here auscultations, such as the lungs or heart, an ultrasound for capturing still images or video, electrocardiogram usually used for obtaining heart readings, sphygmomanometer, or blood pressure unit, blood gas analyzer, blood test strips, and a spirometer to check respiratory and peak flow function. All of these portable devices and technology components, either coupled to, or interfaced with, a device known by the inventor as the 21^{st} Century Doctor's Bag, or similar facsimile thereof, that the healthcare professional carries with them to perform their exam is capable of relaying and transferring the collected patient-encounter information back to the physician at the physicians' center. The physician at the physicians' center receives the images and patient-encounter information from the healthcare professional, and the physician analyses, diagnoses, and instructs the remotely connected healthcare professional on the appropriate treatment and clinical protocols, and concludes the virtual physician visit by recording the virtual physician visit information into the patient's record, like an electronic health record. The healthcare professional implements and performs the treatment plan and protocols as directed by the physician as the information is received. As an example or illustration, the healthcare professional, in communication with the physician following the physician's treatment instruction and protocols uses the otoscope to view the patient's ear, the image is transmitted using a data transfer and communications system comprising of a microprocessor and memory storage that is couple to, or interfaced with a plurality of medical devices and examination equipment, having Internet access, or a telecommunications network system connection to the physician, then the image is viewed by the physician who sees the image and can direct the healthcare professional as they examine the patient. The physician can direct the exam as well as view the patient's image and encounter information in order to render an appropriate diagnosis and treatment plan. The physician may then communicate the diagnosis and treatment plan and protocols back to the healthcare professional and may elect to save or archive either the partial or whole, entire patient encounter information, including digital media, as part of the patient's medical record, like an electronic health record. The 21^{st} Century Doctors Bag, or similar facsimile, and the plurality of technologies comprising of a microprocessor, memory storage, data transfer and communications system, coupled to, or interfaced with, a plurality of medical devices and examination equipment, have the capability to collect, store, transmit and receive data and operational commands as a process of communicating to the physician located at the physicians' center. The healthcare professional may also communicate with the physician a number of ways including but not limited to instant messaging, e-mail, direct conference link or the like.

The virtual house call is like a virtual physician, or clinician, visit in that it permits the physician to direct the patient's plan of care regardless of distance—where the physician is located in one geographic location while the patient resides in another location. The embodiment of the virtual house call involves the alternative delivery of healthcare through the personal contact of a healthcare professional that performs clinical assessments and care to the patient at the patient's location. The patient may be part of a patient population group within a geographical region, or health service area, or it may be someone given privileges who may act on the patient's behalf and who may communicate that they want the patient to be examined by a healthcare professional and the healthcare professional travels to the patient's location to examine the patient. The healthcare professional may also travel to a pharmacy or store to pick up and deliver any of the patient's medications, durable medical equipment, or needed supplies, including but not limited to prescribed medications. The physician at the physicians' center may also issue orders and prescribe medication and durable medical equipment that may be sent to the patient using a postal service, mail service, parcel service, or pharmacy benefit manager contract.

Implementations further provide for a system and method of setting up and using a physicians' center that will receive and transmit information and data to the healthcare professional. The physicians' center is staffed with at least one physician and a plurality of medical and non-medical personnel. Embodiments of this invention include a physician center that can provide treatment, diagnosis, consultation, clinical protocols and algorithms, clinical pathways, disease management, and case management, and any other information to a plurality of healthcare professionals and patient population. One particular implementation of this invention would involve having at least one physicians' center per state such that each state's regulations can be considered and followed by that center. Moreover, the physicians' center includes having or having access to patient's records in a usable form including electronic medical records and electronic health records such that the physician can review and possibly add to the patient's records to aid in patient treatment and diagnosis. The physicians' center is equipped and has access to a plurality of data storage devices or in communication with a plurality of data storage devices such that patient encounter data resulting from a virtual physician visit may be sent via facsimile, common electronic data interchange data sets including Health Language Version (HL) data set (example, HL Version 7), electronic health record data interface, or other electronic means, to participating and non-participating physicians. Some illustrations of a data storage device can be a computer, hard drive, random access memory, tape, digital video disk storage, optical digital jukebox storage, memory storage, microprocessor, compact disc and the like. Data processing and transfer to interconnect the physician, physicians' center, healthcare professional, call center, healthcare advocate, health facility, and business center can occur by use of a central server comprising of a microprocessor, hard drive, memory storage, random access memory, network interfaces, or a plurality thereof.

Implementations of this invention eliminate the need for a patient to travel to a medical facility, like a hospital, to receive efficient quality healthcare, and it provides the patient encounter in the most personalized setting of the patient's home or remote location. Implementations of the present invention use a healthcare professional to travel to the patient's location to examine the patient. The healthcare professional is a person who is trained and in some cases licensed to assess and treat others clinically or medically. Some examples of these professionals include paramedics, emergency medical technicians, registered nurse, nurse practitioners, physicians and physicians' assistants. The healthcare professional may also be trained, licensed, or certified in any number of ways including but not limited to private institutions and government regulatory organizations that train, license, or certify such persons. These healthcare professionals travel with a 21^{st} Century Doctors Bag (as previously defined), or similar facsimile, that contains a number of diagnostic tools, medical equipment and audio/video/data transfer and communications systems that aid in the treatment and diagnosis of the patient.

The 21^{st} Century Doctors Bag, or similar facsimile, has the capability to not only monitor and receive data from a patient, regardless of recumbent or standing position, but also have the capability to transmit that data to a physicians' center. The physician can then receive, retrieve, review, store, and archive the data supplied by the healthcare professional as well as any other data available to allow for the appropriate diagnosis and treatment of the patient.

Implementation of the present invention further provides for the bi-lateral communication between the physician and the healthcare professional. The physician directs and obtains information from the healthcare professional on the patient's condition, including vital readings and diagnostic information. Further this invention supplies the ability for the physician to obtain other data such as medical history of the patient including electronic medical records, billing, drug interactions, and other relevant clinical decision and support applications used by the physician in directing the health professional in assessing the patient's current medical condition, instructing the patient, diagnosing the patient, consulting the patient, and determining the patient's plan of care. Upon completing the embodied implementation of the virtual house call, the physician records the patient episode information into the electronic medical record, or record, and transmits relevant information, like treatment-related orders, back to the healthcare professional using communication systems whereby the healthcare professional executes any transmitted physician orders if applicable. Some examples of communication systems would include the communication of audio, video, and/or electronic data over on-line, public switched telephone network (PSTN), wireless phone, two-way radio, local area network (LAN), wide area network (WAN), integrated service digital network (ISDN), digital subscriber line (DSL), broadband cable, fiber optic network, wireless protocol and the like. The system also provides for the patient to be in communication with someone throughout the virtual physician visit's medical encounter. The person who is in communication with the patient can be clinical personnel or a healthcare advocate and they can coordinate the equipment, route the call to a physician, and help dispatch personnel to the patient.

Furthermore this invention will supply the necessary system and method to virtually eliminate the need for a patient to travel to a medical facility every time the patient requires medical treatment and care; therefore, enabling the embodiment of the new paradigm of the traditional physician house call, or the virtual physician visit through the notion defined by the inventor as the “Connected House Call.” An embodiment of this invention can also allow physicians that are physically impaired to be an associated with the physician's center and allow that physician to practice within the medical field were his physical limitations may have prevented him from doing that in the past.

Yet another embodiment of this invention is having the patient communicate information to the call center. The patient can communicate with the call center by calling the call center via a telephonic call, like a telephone, cellular phone, or Internet protocol phone, to provide general patient information. In addition, the patient may communicate information to the call center via a public switched telephone network, cellular network, Wi-Fi network, wireless network, telephone communication system, or other communications system. General patient information may include information on the patient's current or past health status, such as a chronic or acute illness or disease state, how to treat minor medical situations, questions of past events, subscriber questions and account information, and other member service information. The call center personnel receive the patient's call, triage the patient's call based on information provided by the patient, record the patient's intake information into a reporting system, and input the patient's reported information into the patient's record. The call center routes the patient's call to an appropriate healthcare provider based on the severity of the patient's illness and the intensity of the service required for the patient's situation, and the call center facilitates routing the call to a physician, when appropriate, and notifies the healthcare advocate on the patient's situation. Communication between the call center, the physicians' center, and the patient may occur without a healthcare professional being required to travel to the patient's location for an exam. If the physicians' center receives transmitted patient information by a remote healthcare professional, the physicians' center and the call center record the encounter into the patient's health record, like an electronic health record.

Yet another illustration of this invention is for the physicians' center to support remote patients during an act of bioterrorism, natural disaster, and planning for homeland security and disaster response and preparedness whereby the remote patient, or a remote clinician, contacts the call center, the remote patient, or remote clinician, requires general or immediate medical information, the call center providing the appropriate triage either with general or specific medical information, or connection to qualified clinical and medical personnel. The general medical information may include information on how to treat minor medical situations, answer questions of past events, and the call center personnel recording the encounter into the patient's health record, like an electronic health record. The specific medical information may include information on dispatching healthcare professionals to assist in the direct treatment and care of the trauma patient, or to assist additional medical or clinical personnel in the direct or indirect treatment and support of care to trauma patients or victims of a disaster, and the physician and call center each recording the encounter into the patient's health record, like an electronic health record.

It is a further object of this invention to provide a method that is simple by design and efficient in method and use to enable the health plan to facilitate the provision of health services to emergency medical services by emergency medical service providers, tertiary medical centers, acute care hospitals, and other appropriate emergency medical service providers. The present invention and similar embodiments monitor the continued provision of emergency medical services throughout the patient's stabilization and plan of care and the health plan updates and documents the subscriber's episodic information in the patient's record that is maintained by the invention's embodiment of the health plan.

Illustrative embodiments and modes of operation of the present invention have been described in this specification. The invention which is intended to be protected herein, however, is not to be construed as limited to the particular embodiments disclosed, since these embodiments are to be regarded as illustrative rather than restrictive. Variations and changes may be made by others without departing from the spirit of this invention. Accordingly, it is expressly intended that all such variations and changes which fall within the spirit and scope of the claims be embraced thereby.

### BRIEF DESCRIPTION OF DRAWINGS

Other objects, features, illustrations and embodiments will occur to those skilled in the art from the following description of an embodiment and the accompanying drawings, figures, illustrations in which:

FIG. 1, shows a block diagram of an illustration of the present invention of Patient enrollment.

FIG. 2A-2C, shows a block diagram of an illustration of the present invention of a Patient initiating a call for service.

FIG. 3, shows a block diagram of an illustration of the present invention that a Traveling Health Care Professional may carry with him/her to a patient's exam.

FIG. 4, shows a block diagram of an illustration of the present invention of a Health Care Advocate's function in an inventions embodiment.

FIG. 5, shows a block diagram of an illustration of the present invention showing patient population groups formed in geographical regions.

FIG. 6, shows a block diagram of an illustration of the present invention showing the facilitation of appropriate medical services for a patient.

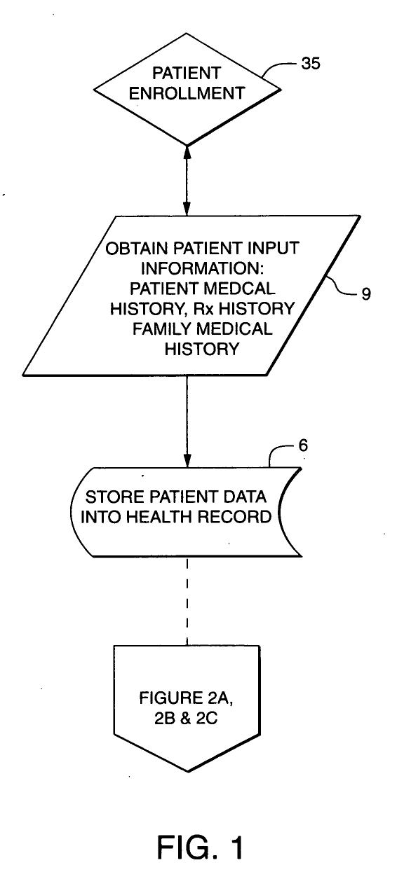

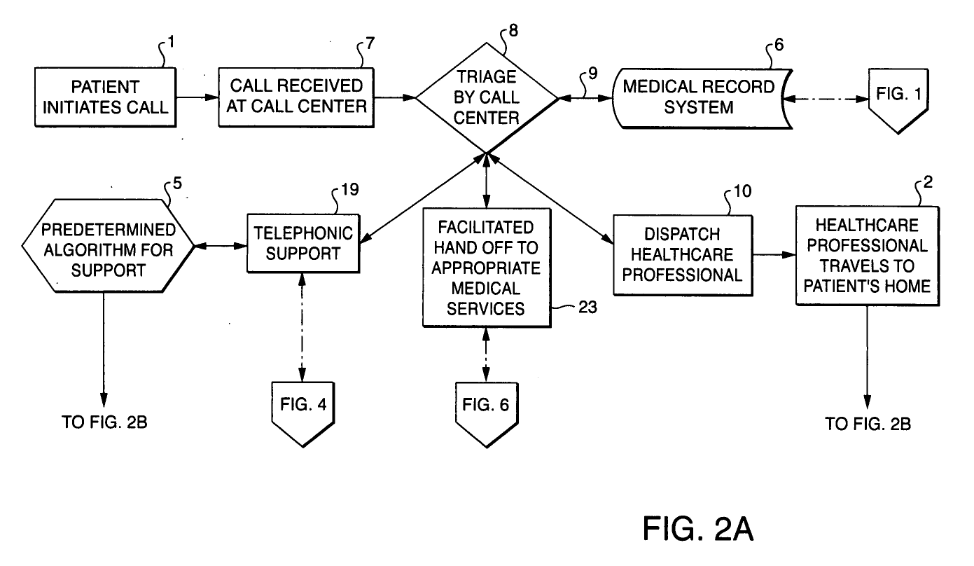

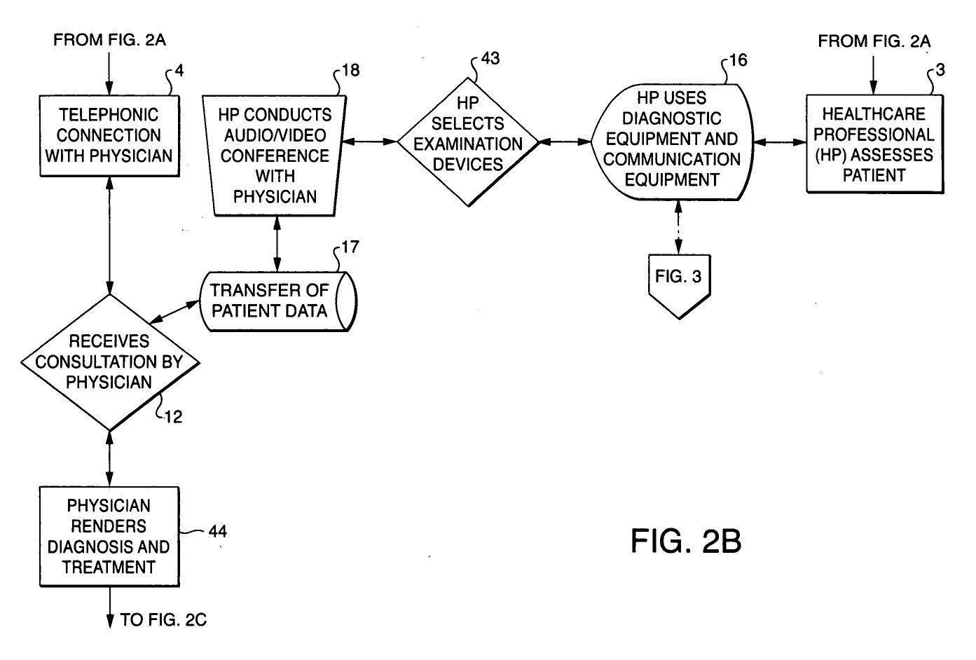

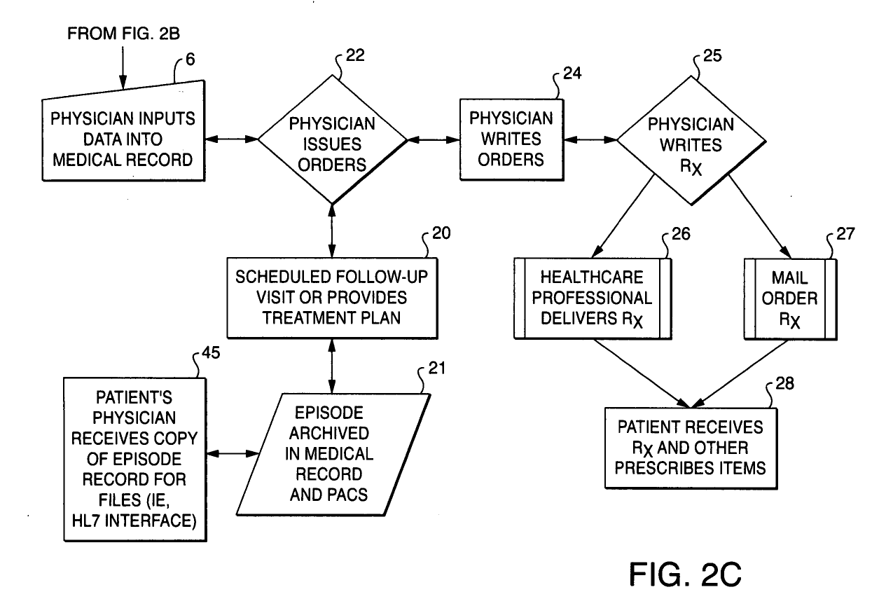

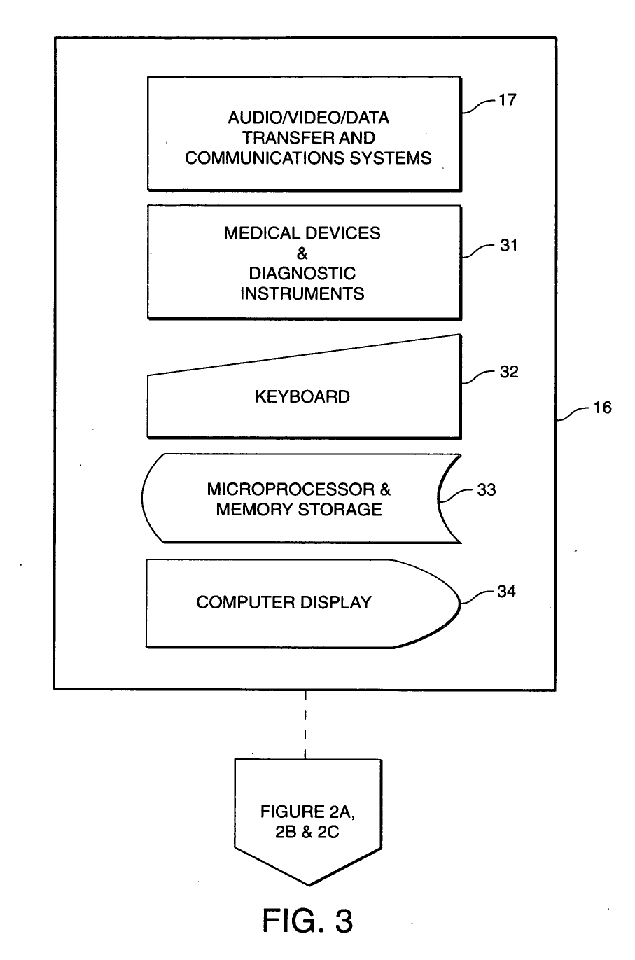

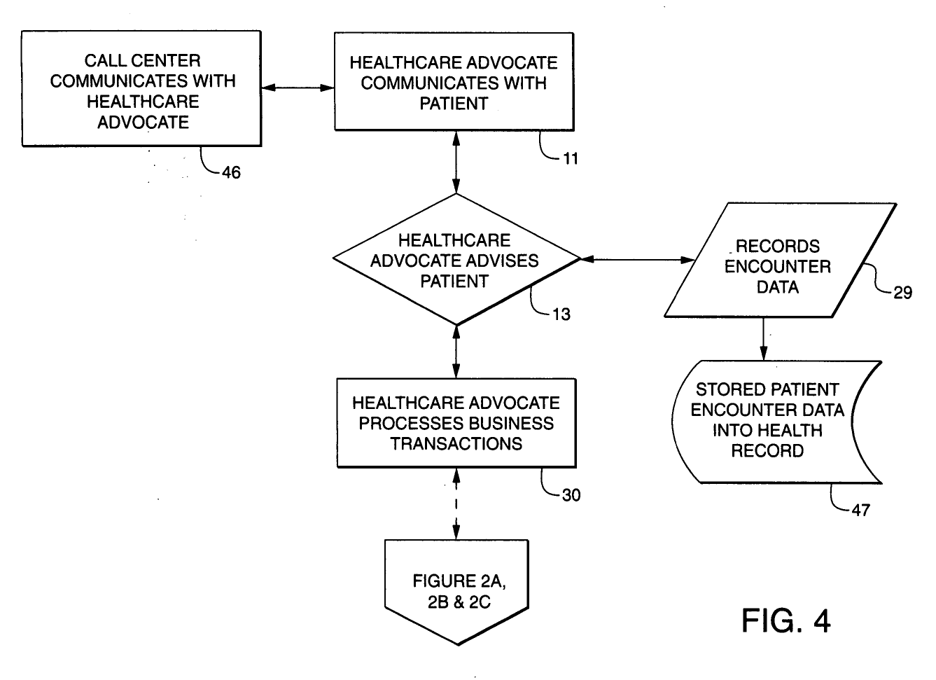

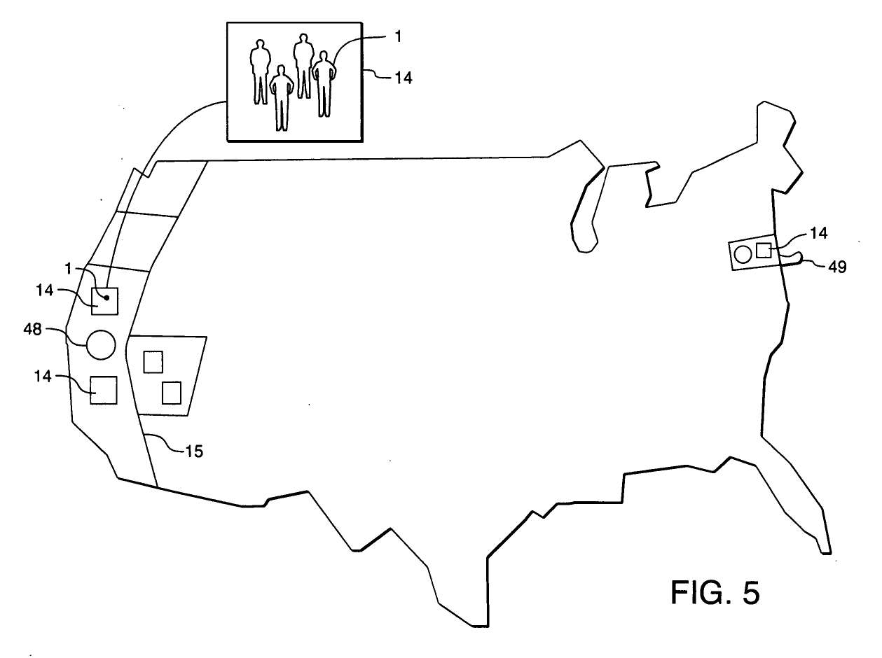

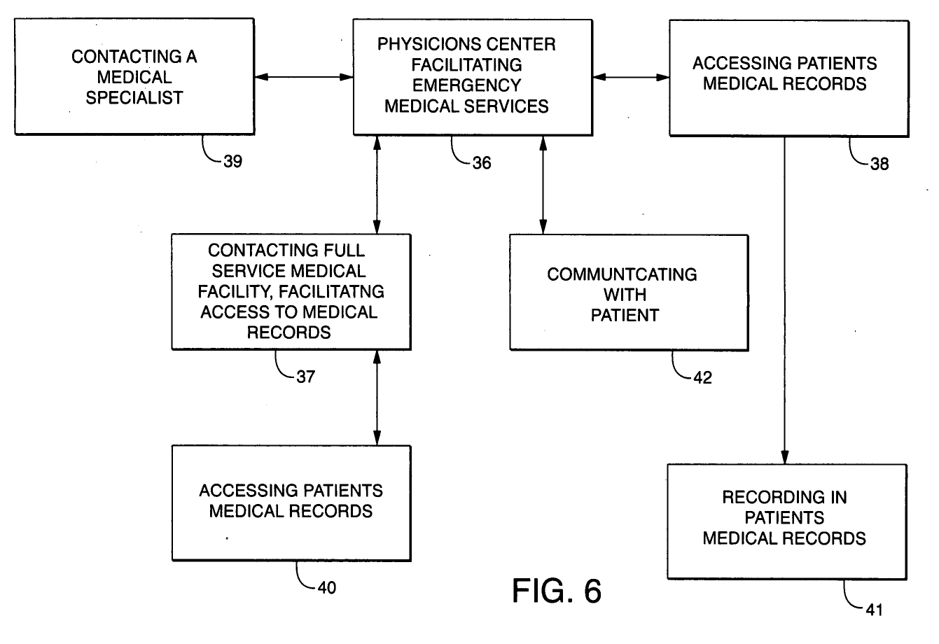

### DETAILED DESCRIPTION

An embodiment of this invention is a method of providing an alternative healthcare delivery system in the form of a virtual house call. In one illustration a patient 1 subscribes to receive member services by enrolling in a health plan 35. During patient enrollment, the patient 1 provides patient-specific information such that a physician-patient relationship is established by obtaining the patient's medical history, prescription history, family medical history, and current medical condition 9 and this information is stored into a medical record system 6. Generally this invention can be enabled by establishing at least a patient population group 14 by forming a patient population group within at least a predetermined geographical region by typically forming a patient population group or groups perhaps located within a state 15, such as Massachusetts 49. Grouping these patients facilitates efficient management of these patients, as well as allowing the traveling Healthcare Professional to reach the patients within a short amount of time. The healthcare delivery system utilizes a medical record system 6 that includes individual information on each patient obtained during patient enrollment within at least the patient population group 14 that is used in the plan of care of the individual patient 1 located at least in the patient population. The patient receives medical care within a reasonable time whereby providing convenient health care to a patient without the patient having to travel to a traditional medical facility. This process and variations of this process is known by the inventor as a virtual house call. Moreover, the virtual house call is initiated by the patient requesting medical services 1.

After enrollment, the patient 1 may access member health services by contacting the call center 7. The patient initiates the call which is routed to the call center 7; the call center receives calls from patients 1 whereby the call center triages 8 the patient's call using online 9 predetermined algorithms and protocols and the patient's medical record 6. The call center appropriately routes the patient's call within the health plan, and the call center records any patient input information into the reporting system 6. The call center 7 routes calls 8 and typically triages calls 5 based on the severity of the illness and intensity of the service for the patient's situation. During the triage process, the call center 7 also notifies a Healthcare Advocate 11. Then the call center connects the patient with the Healthcare Advocate who aids in advising the patient 13 through the encounter as events occur. The patient communicates with the Healthcare Advocate 11, whereby the Healthcare Advocate coordinates the patient's events including advising the patient 13, recording patient input information 29, coordinating the patient encounter information with the appropriate Healthcare Professional and the like. The Healthcare Advocate aids the patient as required, monitors the patient's medical condition, provides following-up care with the patient, maintains records, and processes any business transactions needed 30.

If the call center's triage 8 process determines that a Healthcare Professional needs to be sent to the patient's location, then the call center will dispatch a traveling Healthcare Professional 10 who travels to the patient's location 2 to assess the patient 3 in the patient's environment, such as their home, or remote location and the call center notifies the Physician at the physicians' center of the dispatched Healthcare Professional. Additionally, an embodiment of this invention permits the call center to dispatch a Healthcare Professional to the patient's remote location whereby the remote location may be a battle ground, a natural or manmade disaster area, hotel, office or other remote location. Upon arriving at the patient's location, the Healthcare Professional examines and assesses the patient 3 by utilizing state-of-the-art diagnostic technology and audio/video communication equipment 16 through an embodiment of a portable, mobile doctor's bag, known by the inventor as the 21^{st} Century Doctor's Bag, which is outfitted with equipment that enables the Healthcare Professional to conduct an examination 43 of the patient. During this examination, the Healthcare Professional connects and communicates with a Physician 18 located at the health plan's physicians' center information back concerning the patient's examination, such as vital readings, diagnostic data, and patient input information. The Healthcare Professional communicates with the Physician at the physicians' center using the audio/video and data communication system 16 of the portable, mobile doctor's bag. The Physician can receive, store, and transmit data and information between parties 17. The transfer of the patient input information, vital readings, and diagnostic data 17 taken during the course of the Healthcare Professional's examination of the patient occurs over a telecommunications connection, such as over the Internet, or direct point-to-point connection. The telecommunications connection may include a public switch telephone network, satellite communications, wireless connection, Wi-Fi connection, 802.11 connection, or Internet Protocol connection such that the Physician remotely examines the information and consults the patient 12, and renders a diagnosis and treatment plan 44.based on the patient's situation and in lieu of their record. Upon rendering the Physician's orders and/or directives 22, the Healthcare Professional administers the Physician's direction and treatment plan to the patient through the use of the audio/video communication system 16. Upon the issuance of the physician orders, the Physician writes the orders 24 and may write a prescription 25 that may be delivered and administered by the Healthcare Professional 26 or mailed 27 using a parcel delivery service, pharmacy benefit manager program, or the United States Postal system.

Once the Physician issues the orders for the patient, the physicians' center schedules a follow-up patient visit or a patient treatment plan 20 as directed by the Physician. Upon the physicians' center completing the patient's next scheduled appointment and any subsequent treatment services 21, the patient's physician, whether out-of-network or the health plan's designated physician for the patient, receives a copy of the patient encounter 45 of the health plans's encounter through the transmission and receipt via facsimile or via electronic data interchange using standard data interface protocols, such as Health Language (HL) Versions (example, HL&7).

Illustrations of this invention include having a physicians' center 48 wherein each physicians' center supports and communicates to at least a patient 1 within a patient population group 14. Moreover, the physicians' center 48 supports a plurality of patient population groups perhaps within a state 15 remotely treating patients as patients have events and as events occur; the physicians' center can support a plurality of patient population groups in multiple states. The physicians' center 48 is staffed with at least a Physician.

Embodiments of this invention also include supplying the patient 1 with access to a traveling Healthcare Professional 10 that travels to the patient's location 2 within the predetermined geographical region 14 who is able to assess and provide a physical examination 3 and administer treatment 44 to the patient as required. The Healthcare Professional travels to the patient's location 2, arrives at the patient's location, communicates and assesses the patient 3, triages the patient, evaluates the patient, accesses the patients records 6, collects patient input information, and examines the patient using a plurality of diagnostic equipment and audio/video communication systems 16 by selecting those devices and equipment most appropriate for the patient's examination 43. The diagnostic equipment that the traveling Healthcare Professional has available to him/her is used to collect, store, and transmit information 17 back to the call center and Physician 18 such that the Physician can consult the Healthcare Professional 12 and render a diagnoses 44 and respond back to the Healthcare Professional so that he/she can treat the patient as instructed 12. Typically, in one variation of this invention the Healthcare Professional and Physician are storing and exchanging data via a networked communications system or computer system. Some equipment available for selection by the Healthcare Professional includes but not limited to an: otoscope, stethoscope, ultrasound, electrocardiogram, thermometer, device that measures blood pressure, a blood gas analyzer, a spirometer, digital camera, digital video camera, a weight scale, a device that tests urine, a device that tests feces, a device that tests blood, device that collects patient vital signs, a device that measures peak flow, a portable computer, memory storage device, diagnostic software, a data storage device, a display monitor, communications equipment, and other like equipment 31, 32, 33, 34. A collection of this equipment may be typically known by the inventor as a 21^{st} century doctor's bag. The Healthcare Professional collects patient vital reading information, which is information from the examination, and records patient input information and observations and readings from the diagnostic equipment 16. In conjunction with the exam the Healthcare Professional communicates and transmits the patient input information and the patient vital reading information to the physicians' center for analysis thereby having an audiovisual conference with the physician 18. The Healthcare Professional in evaluating the patient can select and use examination equipment 43 and can also use a device comprised of at least a display unit for viewing multimedia which includes a microprocessor having a memory storage device coupled to a data communications system, medical devices, and vital measurement instruments that networks the Healthcare Professional with the physicians' center. At that time the Physician analyzes the information, communicates, and instructs the Healthcare Professional, the Physician is directing the examination at least during the Healthcare Professional's patient exam and evaluation of the patient. The Physician at least accesses the patient's records, reviews the patient's past medical history, evaluates the family medical history, learns of patient's past and present medications, and investigates if the patient has experienced any past allergic reactions, including if the patient has experienced any past drug interactions. During the visit the Physician assesses the patient's current medical condition, the Physician instructs the patient, the Physician diagnoses the patient, and the Physician consults the patient 12. This invention allows the patient to be examined and treated in a reasonable time frame in an environment that may not be in a traditional medical office setting. The Physician makes his/her diagnosis and treatment suggestions using the information he/she has received from the patient, and the Healthcare Professional as well as any other medical data the Physician has available from the patient. Strategically, the Physician determines the best plan of care for the patient then records the patient episode in the medical record and transmits treatment information back to the Healthcare Professional. Following the Physician's direction the Healthcare Professional then treats the patient using the Physician's instructions and directions, therein providing the patient with a virtual house call. At the same time, the physician prescribes medication if required 25, checks for drug-to-drug interactions and for contraindications, writes orders 24, prescribes durable medical equipment if required, transmits the prescription to a pharmacy if required, and, accesses the patient's records, revises the patient's records using the patient input information and diagnostic data, associated clinical notes, Physician orders if required, and treatment information. In addition, the Healthcare Professional may travel to a location to collect 26 and deliver to the patient 28 the prescribed medications or durable medical equipment which may be sent via other means such as by delivery service, parcel, or United States postal service 27.

In another illustration of this invention where a Healthcare Professional does not travel to the location of the patient, as an alternative, the patient's call is received by the call center 7 and networked 8 for telephonic support 19 using predetermined algorithms where the Physician communicates directly with the patient 4. The Physician communicates with the patient by at least accessing the patient's record, reviewing the patient's past medical history, evaluating family medical history, learning of the patient's past medication history, learning of the patient's present medications, investigating if the patient has experienced any past allergic reactions, including if the patient has experienced any past drug interactions in order for the Physician to assess and consult 12 the patient's current medical condition. Then after conducting the evaluation, the Physician diagnoses and instructs the patient 44, by issuing orders which directs the patient with any diagnosis and treatment information. The Physician considers all the information that is collected during the patient encounter and consults with the patient; the Physician records the encounter into the patient's record 6 and issues orders to the patient 22 by communicating the best plan of care for the patient. Subsequent to the Physician issuing the patient orders, the patient may be scheduled for a follow-up visit or ongoing treatment plan 20 along with the recommended care instructions. The physician notes, recorded encounter data, orders, collected encounter data including diagnostic images and vital data are all archived into the medical record system and picture archiving and communication system for reference at another time 21, therein recording the entire patient visit and clinical services rendered. The Physician in the normal course of his diagnosis can prescribe medication if required, check for drug-to-drug interactions, check for contraindications, prescribe durable medical equipment if required 24, transmit the prescription to a pharmacy 25 if required, and, access the patient's records, revise the patient's records using the patient input information, diagnostic data, associated clinical notes, physician orders if required, and treatment information 22.

In yet another embodiment of this invention is a method of providing health care in the form of a facilitated hand off to an appropriate medical service provider 23. After it is determined that the patient is in need of appropriate medical care, like emergency medical services, the physicians' center facilitates emergency medical services to the patient 36. This is performed at least by the call center contacting a full service medical facility 37, and advising the full service facility of the need for immediate medical patient care, perhaps facilitating ambulance transportation to the full service facility, such as a hospital. The center also can facilitate access to the patient's information 38 by communicating the patient's collected and evaluated medical information to at least a full service facility. The center can also facilitate access to the patient's information by contacting a medical specialist 39 regarding the patient's medical condition, and communicating with the medical specialist regarding the patient's medical condition. This facilitation allows for more effective and efficient patient treatment as the patient is received at the emergency medical care establishment. The service provider of the emergency medical service center is provided access to the patient's records 40 by the physicians' center. The physicians' center communicates any pertinent information to the appropriate medical service provider, such as a full service medical provider or emergency medical service provider, and revises the patient's records using the patient episodic information, such as diagnostic data, associated clinical notes, physician orders if required, and treatment information the patient obtains during the emergency medical service encounter, full service medical facility, or specialty encounter. The service personnel involved during these encounters record the patient episode information into the patient's record 41 and communicate with the patient as needed 42.

Another embodiment of this invention enables a method of providing health care through the interaction of the Healthcare Advocate 11. This method includes facilitating a patient's call to the Healthcare Advocate. The call center communicates with the Healthcare Advocate 46, and then the Healthcare Advocate communicates with the patient 11. The Healthcare Advocate facilitates, coordinates, monitors, and aids in guiding, answering and advising the patient 13. The Healthcare Advocate may provide follow-up services to the patient as necessitated by the patient's needs by communicating with the patient and providing personal assistance telephonically to the patient. The Healthcare Advocate accesses the patient's records, revises the patient's records 29, stores any interactions in the patients medical record 47 using the patient input information along with associated clinical notes in order to document any information provided by the patient, such as executing physician orders when required, as well as relevant treatment information and business transactions 30. A hard copy or electronic copy of the patient's record, such as a record of the virtual house call encounter, may be provided to the patient's physician even if they are outside of the alternative healthcare delivery system, such as an out-of-network physician 45.

In concise summary, the Healthcare Professional, Physician, call center personnel, and Healthcare Advocate may access, update, and append, transfer the patients records to appropriate medical service providers including out-of-network physicians and providers, and archive the patient-encounter data into an electronic health record using a plurality of technologies having at least a microprocessor including a memory storage device that may be coupled to audio, video, and data communications systems, medical devices, communications equipment and examination equipment used in the assessment and communication of the patient. The alternative healthcare delivery system envisioned by the inventor utilizes a method of archiving and managing patient data using an electronic medical record whereby the patient data is inputted, collected, sent to, and accessible from a central data storage device. This archived patient encounter data is accessible from a central data storage device over a telecommunications network system. The telecommunications network system can be Internet. The central data storage device can be a server having a microprocessor, database, including a memory storage unit that is in communication with a LAN, WAN, virtual private network, communications system, Internet network connection, telecommunications network connection or the like, including collecting and storing information from medical devices, vital measurements, telecommunications network connections.

Other embodiments of this invention include a method of using a call center to provide disease state management that utilizes at least the formed patient population group 14 within at least a predetermined geographical region 15. The call center communicates with a plurality of patients 14 by sending or receiving information to the patient population groups by using any one of several available technologies such as a telecommunications network, a telephonic device, a telecommunications device for making phone calls, a telecommunications device for receiving phone calls and the like in order to collect information from each patient. The call center utilizes surveys to gather data from the patients using predetermined questions such as to measure the quality of life of the patient, plurality of patients and patient population groups, perhaps using a clinical algorithm that measures their health status or present disease condition. Examples of these predetermined questions that patients may receive or send would be to collect and assess patient information such as body weight, vital sign data, general patient input information, drug compliance information, treatment compliance, and general health concerns. The call center collects this patient information and records it into a database application. The storing and collection of the inputted patient information is archived and stored on a server having at least a microprocessor including a memory storage device that enables the administrative function of compiling, analyzing and normalizing the collected information and data.

What is claimed is:

1. A method of providing healthcare in the form of a virtual house call, the method comprising the steps of:
establishing at least a patient population group;
forming at least a patient population group within at least a predetermined geographical region;
utilizing a medical record system that includes individual information on each patient within at least the patient population group that is used in the plan of care of the individual patient within at least the patient population group;
establishing a physician-patient relationship with each individual patient within the patient population group;
establishing a call center, receiving calls from patients, triaging patient calls based on information provided by the patient, recording patient intake information into a reporting system, inputting patient reported information into a patient's record, routing calls based on the severity and intensity of the patient's situation, arranging and dispatching a healthcare professional to travel to the patient's location, notifying a healthcare advocate, notifying a physician of the impending patient visit;
providing the patient with access to the healthcare advocate, the healthcare advocate communicating with the patient, recording patient input information, coordinating the patient encounter information with the appropriate healthcare professional, aiding the patient as required, monitoring the patient's medical condition, providing following-up care with the patient;
establishing a physicians' center, each physicians' center supporting at least the patient population group, each physicians' center communicating to at least the patient population group, staffing the physicians' center with at least a physician;
providing the patient with access to a traveling healthcare professional that travels to the patient's location within the predetermined geographical region;
the healthcare professional traveling to the patient's location, arriving at the patient's location, communicating with the patient, triaging the patient, evaluating the patient, assessing the patient, collecting patient input information, examining the patient using a plurality of diagnostic equipment, collecting patient vital reading information, collecting information from the diagnostic equipment, transmitting the patient input information, transmitting the patient vital reading information, communicating with the physicians' center, transmitting the patient information to the physicians' center for analysis, evaluating the patient using a device comprised of at least a display unit for viewing multimedia, a microprocessor having a memory storage device coupled to an audio video data communications system, medical devices, and vital measurement instruments;
networking the healthcare professional with the physicians' center, the physicians' center having at least a physician to evaluate the patient input information, patient vital readings, patient diagnostic information, the physician instructing the healthcare professional, directing the examination at least during the healthcare professional's patient exam and evaluation of the patient, the physician at least accessing the patient's records, reviewing the patient's past medical history, evaluating family medical history, learning of patient's past and present medications, investigating if the patient has experienced any past allergic reactions, including if the patient has experienced any past drug interactions, the physician assessing the patient's current medical condition, the physician instructing the patient, the physician diagnosing the patient, the physician consulting the patient, the physician determining the plan of care for the patient, the physician recording the patient episode in the medical record, the physician transmitting treatment information back to the healthcare professional, the healthcare professional treating the patient using the physician's instructions, therein providing the patient with a virtual house call;
prescribing medication if required, checking for drug-to-drug interactions, checking for contraindications, prescribing durable medical equipment if required, transmitting the prescription to a pharmacy if required; and,
accessing the patient's records, revising the patient's records using the patient input information, diagnostic data, associated clinical notes, physician orders if required, and treatment information.

2. The method of claim 1 wherein a plurality of patient population groups is within a state.

3. The method of claim 1 wherein the patient population group is within a state.

4. The method of claim 1 wherein a plurality of patient population groups is within the United States of America.

5. The method of claim 1 wherein the physicians' center supporting a plurality of patient population groups is within the same state as the plurality of patient population groups.

6. The method of claim 1 wherein the physicians' center supporting a patient population group is within the same state as the patient population group.

7. The method of claim 1 wherein the call center notifies the healthcare advocate.

8. The method of claim 1 where in the physician facilitates routing the patient's plan of care to appropriate healthcare providers.

9. The method of claim 1 wherein the physicians' center contains a transmitting device in addition to a receiving device.

10. The method of claim 1 wherein the healthcare professional has a transmitting device in addition to a receiving device.

11. The method of claim 1 wherein the physicians' center contains a data storage device.

12. The method of claim 1 wherein the healthcare professional has a data storage device.

13. The method of claim 1 wherein interconnecting the healthcare professional with the physicians' center comprises a computer having at least a display unit for viewing multimedia, a microprocessor having a memory storage device coupled to an audio video and data communications system.

14. The method of claim 1 wherein interconnecting the healthcare professional with the physicians' center is wireless.

15. The method of claim 1 wherein interconnecting the healthcare professional with the physicians' center is a telephone.

16. The method of claim 1 wherein interconnecting the healthcare professional with the physicians' center is a digital subscriber line.

17. The method of claim 1 wherein interconnecting the healthcare professional with the physicians' center is a local area network

18. The method of claim 1 wherein interconnecting the healthcare professional with the physicians' center is a wide area network.

19. The method of claim 1 wherein interconnecting the healthcare professional with the physicians' center is an integrated service digital network.

20. The method of claim 1 wherein interconnecting the healthcare professional with the physicians' center is a broadband cable network.

21. The method of claim 1 wherein interconnecting the healthcare professional with the physicians' center is a satellite connection

22. The method of claim 1 wherein interconnecting the healthcare professional with the physicians' center is a wireless network.

23. The method of claim 1 wherein interconnecting the healthcare professional with the physicians' center is a fiber optic communication.

24. The method of claim 1 wherein the time from receiving communication from the patient to the healthcare professional examining and evaluating the patient is set to a predetermined time.

25. The method of claim 1 wherein providing health care is twenty-four hours a day seven days a week fifty-two weeks a year.

26. The method of claim 1 wherein the health care is emergency care.

27. The method of claim 1 wherein the health care is urgent care.

28. The method of claim 1 wherein the health care is routine care.

29. The method of claim 1 wherein the health care is case management.

30. The method of claim 1 wherein the health care is preventative care.

31. The method of claim 1 wherein the health care is wellness care.

32. The method of claim 1 wherein the health care is hospice care.

33. The method of claim 1 wherein the health care is non-emergent care.

34. The method of claim 1 wherein the health care is chronic care.

35. The method of claim 1 wherein the health care is non-acute care.

36. The method of claim 1 wherein the healthcare professional is a paramedic.

37. The method of claim 1 wherein the healthcare professional is a Physician.

38. The method of claim 1 wherein the healthcare professional is a Registered Nurse.

39. The method of claim 1 wherein the healthcare professional is an Emergency Medical Technician.

40. The method of claim 1 where in the healthcare professional is a certified technician.

41. The method of claim 1 where in the healthcare professional is a trained technician.

42. The method of claim 1 wherein the diagnostic equipment is an otoscope.

43. The method of claim 1 wherein the diagnostic equipment is a stethoscope.

44. The method of claim 1 wherein the diagnostic equipment is an ultrasound.

45. The method of claim 1 wherein the diagnostic equipment is an electrocardiogram.

46. The method of claim 1 wherein the diagnostic equipment is a thermometer.

47. The method of claim 1 wherein the diagnostic equipment is a device that measures blood pressure.

48. The method of claim 1 wherein the diagnostic equipment is a blood gas analyzer.

49. The method of claim 1 wherein the diagnostic equipment is a spirometer.

50. The method of claim 1 wherein the diagnostic equipment is a digital camera.

51. The method of claim 1 wherein the diagnostic equipment is a digital video camera.

52. The method of claim 1 wherein the diagnostic equipment is a weight scale.

53. The method of claim 1 wherein the diagnostic equipment is a device that tests urine.

54. The method of claim 1 wherein the diagnostic equipment is a device that tests feces.

55. The method of claim 1 wherein the diagnostic equipment is a device that tests blood.

56. The method of claim 1 wherein the diagnostic equipment is a device that collects patient vital signs.

57. The method of claim 1 wherein the diagnostic equipment is a device that measures peak flow.

58. The method of claim 1 wherein the diagnostic equipment is a portable computer.

59. The method of claim 1 wherein the diagnostic equipment is a device comprised of at least a microprocessor having a memory storage device.

60. The method of claim 1 wherein the diagnostic equipment is diagnostic software.

61. The method of claim 1 wherein the patient's record resides on a central data storage device.

62. The method of claim 1 wherein the patient's record is an electronic medical record.

63. The method of claim 1 wherein the collected information is a patient's medical record.

64. The method of claim 1 wherein the patient's record is an electronic health record.

65. The method of claim 1 wherein the collected information is a patient's health record.

66. The method of claim 1 wherein the collected information is patient input information.

67. The method of claim 1 wherein the patient's location is at a home.

68. The method of claim 1 wherein the patient's location is at an office.

69. The method of claim 1 wherein the patient's location is at a hotel.

70. The method of claim 1 wherein the patient's location is at a disaster area.

71. The method of claim 1 wherein the patient's location is at a battlefield.

72. The method of claim 1 wherein the patient's location is a remote location.

73. The method of claim 1 wherein the healthcare professional travels to a location to obtain and deliver to the patient any medical supplies.

74. The method of claim 1 wherein the healthcare professional provides to the patient prescribed medications.

75. A method of providing health care in the form of a telephonic patient visit comprising the steps of:
establishing at least a patient population group;
forming at least a patient population group within at least a predetermined geographical region;
utilizing a medical record system that includes individual information on each patient within the patient population using the medical record in the plan of care of the individual patient within the patient population group;
establishing a physician-patient relationship with each individual patient within the patient population group;
establishing a telephonic call center, receiving telephonic calls from patients, triaging patient telephonic calls based on information provided by the patient, recording patient intake information into a reporting system, inputting patient reported information into the patient's record, routing telephonic calls based on the severity and intensity of the patient's situation, facilitating the patient's call to the physician, notifying the healthcare advocate;
providing the patient with access to a healthcare advocate, the healthcare advocate communicating with the patient, recording patient input information, coordinating the patient encounter information with the appropriate healthcare professional, aiding the patient as required, monitoring the patient's medical condition, providing following-up care with the patient;
establishing a physicians' center, each physicians' center supporting at least a patient population group, each physicians' center communicating to at least a patient population group, staffing the physicians' center with at least a physician;
networking a telephonic call with the physicians' center, the physician center having at least a physician to evaluate the patient's input information, the physician at least accessing the patient's record, reviewing the patient's past medical history, evaluating family medical history, learning of the patient's past medication history, learning of the patient's present medications, investigating if the patient has experienced any past allergic reactions, including if the patient has experienced any past drug interactions, the physician assessing the patient's current medical condition, the physician instructing the patient, the physician diagnosing the patient, the physician consulting the patient, the physician determining the plan of care for the patient, the physician recording the patient episode in the medical record, therein providing the patient with a telephonic patient visit;
prescribing medication if required, checking for drug-to-drug interactions, checking for contraindications, prescribing durable medical equipment if required, transmitting the prescription to a pharmacy if required; and,
accessing the patient's records, revising the patient's records using the patient input information, diagnostic data, associated clinical notes, physician orders if required, and treatment information.

76. The method of claim 75 wherein a plurality of patient population groups is within a state.

77. The method of claim 75 wherein the patient population group is within a state.

78. The method of claim 75 wherein the plurality of patient population groups is within the United States of America.

79. The method of claim 75 wherein the physicians' center supporting a plurality of patient population groups is within the sane state as the plurality of patient population groups.

80. The method of claim 75 wherein the physicians' center supporting a patient population group is within the same state as the patient population group.

81. The method of claim 75 wherein the call center notifies the healthcare advocate.

82. The method of claim 75 where in the physician facilitates routing the patient's plan of care to appropriate healthcare providers.

83. The method of claim 75 wherein the physicians' center contains a transmitting and a receiving device.

84. The method of claim 75 wherein the physicians' center contains a data storage device.

85. The method of claim 75 wherein the patient's record resides on a central data storage device.

86. The method of claim 75 wherein the patient's record is an electronic medical record.

87. The method of claim 75 wherein the collected information is a patient's medical record.

88. The method of claim 75 wherein the patient's record is an electronic health record.

89. The method of claim 75 wherein the collected information is a patient's health record.

90. The method of claim 75 wherein the collected information is patient input information.

91. The method of claim 75 wherein the patient communicates using a communication device.

92. The method of claim 75 wherein the patient communicates using a telephone.

93. The method of claim 75 wherein the patient communicates using a telephonic device.

94. The method of claim 75 wherein the patient communicates using a data communications device.

95. The method of claim 75 wherein the patient communicates using a voice over Internet protocol communication device.

96. The method of claim 75 wherein the patient communicates using a cellular communication device.

97. The method of claim 75 wherein the patient communicates using a satellite communication device.

98. The method of claim 75 wherein the patient communicates using a satellite phone.

99. The method of claim 75 wherein the patient communicates using a video communication device.

100. The method of claim 75 wherein the healthcare professional travels to a location to obtain and deliver to the patient any medical supplies.

101. A method of providing health care in the form of a facilitated hand off to a medical service provider comprising the steps of:
establishing at least a patient population group;
forming a patient population group within at least a predetermined geographical region;
utilizing a medical record system that includes individual information on each patient within the patient population group that is used in the plan of care of the individual patient within the patient population group;
establishing a physician-patient relationship with each individual patient within the patient population group;
establishing a call center, receiving calls from patients, triaging patient calls based on information provided by the patient, recording patient intake information into a reporting system, inputting patient reported information into the patient's record, routing calls based on the severity and intensity of the patient's situation, facilitating emergency medical services to the patient, notifying a healthcare advocate, notifying the physician;
providing the patient with access to the healthcare advocate, the healthcare advocate communicating with the patient, recording patient input information, coordinating the patient encounter information with the appropriate healthcare professional, aiding the patient as required, monitoring the patient's medical condition, providing following-up care with the patient;
establishing a physicians' center, each physicians' center supporting at least a patient population group, each physicians' center communicating to at least a patient population group, staffing the physicians' center with at least a physician;
the physician' center facilitating emergency medical services to the patient, contacting at least the full service medical facility to facilitate access to the patient's information, communicating the patient's collected and evaluated medical information to at least a full service facility, contacting a medical specialist regarding the patient's medical condition, communicating with the medical specialist regarding the patient's medical condition;
providing the patient with access to a traveling healthcare professional that travels to the patient's location within at least the predetermined geographical region;
the healthcare professional traveling to the patient's location, arriving at the patient's location, and communicating with the patient, triaging the patient, evaluating the patient, assessing the patient, collecting patient input information, examining the patient using a plurality of diagnostic equipment, collecting patient vital reading information, collecting information from the diagnostic equipment, facilitating emergency medical services to the patient, communicating with the physicians' center;
accessing the patient's records, revising the patient's records using the patient input information, diagnostic data, associated clinical notes, physician orders if required, treatment information, obtaining emergency medical service patient episode information, and obtaining the full service medical facility patient episode information,
recording the emergency medical service patient episode information into the patient's record;
recording the full service medical facility patient episode information into the patient's record; and,
recording the medical specialist patient episode information into the patient's record.

102. The method of claim 101 wherein the plurality of patient population groups is within a state.

103. The method of claim 101 wherein the patient population group is within a state.

104. The method of claim 101 wherein the plurality of patient population groups is within the United States of America.

105. The method of claim 101 wherein the physicians' center supporting a plurality of patient population groups is within the same state as the plurality of patient population groups.

106. The method of claim 101 wherein the physicians' center supporting a patient population group is within the same state as the patient population group.

107. The method of claim 101 wherein the patient's record resides on a central data storage device.

108. The method of claim 101 wherein the patient's record is an electronic medical record.

109. The method of claim 101 wherein the collected information is a patient's medical record.

110. The method of claim 101 wherein the patient's record is an electronic health record.

111. The method of claim 101 wherein the collected information is a patient's health record.

112. The method of claim 101 wherein the collected information is patient input information.

113. A method of providing health care through the interaction of a healthcare advocate, the method comprising the steps of:
establishing at least a patient population group;
forming at least a patient population group within at least a predetermined geographical region;
utilizing a medical record system that includes individual information on each patient within the patient population group using the medical record in the plan of care of the individual patient within the patient population group;
establishing a physician-patient relationship with each individual patient within at least the patient population group;
establishing a call center, receiving calls from patients, triaging patient calls based on information provided by the patient, recording patient intake information into a reporting system, routing calls based on the severity and intensity of the patient's situation, facilitating the patient's call to the healthcare advocate; and,
the healthcare advocate facilitating, coordinating, monitoring, and aiding in follow-up services to the patient as necessitated by the patient's needs, communicating with the patient, providing personal assistance telephonically to the patient, the healthcare advocate accessing the patient's records, revising the patient's records using the patient input information along with associated clinical notes, documenting any information provided by the patient, executing physician orders if required, including relevant treatment information.

114. A method for archiving patient-encounter data into an electronic health record comprising the steps of:
establishing a telecommunications connection with a healthcare professional;
receiving patient encounter data collected by the healthcare professional using a plurality of technologies having at least a microprocessor including a memory storage device, coupled to audio, video, and data communications systems, medical devices, and examination equipment used in the assessment of the patient;
transferring the collected patient encounter data to a central data storage device; and,
accessing archived patient encounter data from the central data storage device over a telecommunications network system.

115. The method of claim 114 wherein the telecommunications network system is the Internet.

116. The method of claim 114 wherein the electronic health record is an electronic medical record.

117. The method of claim 114 wherein the central data storage device is a server having a microprocessor, database, including a memory storage that is in communication with, a LAN, WAN, virtual private network, communications system, Internet network connection, telecommunications network connection.

118. A method of transferring electronic health record information having patient-encounter data comprising the method of:
using a device comprised of at least a microprocessor having a memory storage device, coupled to a data communications systems, including medical devices, vital measurement instruments, telecommunications network connection;
communicating with a server having a microprocessor, database, including a memory storage that is in communication with, a LAN, WAN, virtual private network, communications system, Internet network connection, telecommunications network connection;
transferring patient-encounter data resulting from a virtual house call;
transferring patient-encounter data resulting from a telephonic patient visit;
transferring patient-encounter data resulting from a facilitated handoff to emergency medical services;
transferring the electronic health record to an out-of-network physician, physician practice, hospital, clinic, health service organization using a common, standard data set in the data communication;
transferring the electronic health record to an out-of-network physician, physician practice, hospital, clinic, or health service organization using electronic facsimile; and,
transferring the electronic health record to a health service organization including an out-of-network physician, physician practice, hospital, clinic, using digital transmission.

119. The method of claim 118 wherein the common, standard data set for data transmission is one of several American National Standards Institute ANSI accredited Standards Developing Organizations operating in the healthcare arena for a particular healthcare domain such as pharmacy, medical devices, imaging or insurance (claims processing) transactions.

120. A method of using a call center to provide disease state management comprising the steps of:
forming at least a patient population group within at least a predetermined geographical region;
using a telecommunications network, using a telephonic device, using a telecommunications device for making phone calls, using a telecommunications device for receiving phone calls;
contacting a plurality of patient population groups, contacting a population group, questioning patients with predetermined questions, measuring the quality of life of the plurality of patient population groups, measuring the quality of life of the patient population group;
contacting a plurality of patients with clinical algorithms that measure their health status of the patients;
contacting a plurality of patients for drug compliance;
contacting a plurality of patients for treatment compliance;
recording the collected information into a database application;
storing the collected information on a server having at least a microprocessor including a memory storage device;
contacting plurality of patients to acquiring information on their body weight;
contacting plurality of patients to collect patient vital sign date;
contacting a plurality of patients to collect patient input information;
analyzing the collected information; and,
using a plurality of automated telecommunications technology, including auto dialing technologies, interactive voice response systems, technology having a microprocessor including a memory storage device that automatically dials patients for the call center.

121. A method of using a call center to provide organizational services comprising the steps of:
establishing at least a patient population group;
forming at least a patient population group within at least a predetermined geographical region;
using a telecommunications network, using of a telephone in communication with a telecommunications device for making then receiving phone calls, contacting people, receiving input information;
recording the collected information into a database application, storing the collected information on a server having at least a microprocessor including a memory storage device, contacting a patient with a questionnaire, analyzing the collected information, normalizing the collected information; and,
using a plurality of automated telecommunications technologies, including, an auto dialing technologies, interactive voice response systems, having a microprocessor including a memory storage device that automatically dials predetermined or random population of people for the call center.
